(19) United States
(12) Patent Application Publication
(10) Pub. No.: US 2005/0048501 A1
(43) Pub. Date: Mar. 3, 2005

(54) Method and apparatus for detection or identification of DNA

(76) Inventors: Robert Marcus Corn, Madison, WI (US); Terry Thomas Goodrich, Madison, WI (US); Hye Jin Lee, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 10/653,365

(22) Filed: Sep. 2, 2003

(51) Int. Cl. C12Q 1/68; C12P 19/34

(52) U.S. Cl. 435/6; 435/91.2

(57) ABSTRACT

DNA is detected using complementary RNA probes and an enzyme that attacks and hydrolyzes the RNA probes only when it has hybridized with target DNA. A low concentration of target DNA can therefore successively hydrolyze a larger amount of RNA whose loss may then be detected to indirectly determine the presence of the target DNA.

### CROSS-REFERENCE TO RELATED APPLICATIONS

### STATEMENT REGARDING FEDERALLY SPONSORED RESEARCH OR DEVELOPMENT

### BACKGROUND OF THE INVENTION

The present invention relates to instruments and methods for the detection and/or identification of DNA.

The detection and identification of oligonucleotides, genomic and PCR (polymerase chain reaction) amplified DNA is important in a wide variety of applications ranging from basic research and medical diagnostics to the detection of bioterrorism. One commonly used method for the detection and identification of DNA makes use of “gene chips”, in which ssDNA molecules are immobilized onto a substrate. These probe molecules may be arranged at the intersections of a rectangular grid over the surface of the substrate, each with the ability to hybridize to their complementary target DNA sequence. For most applications the DNA target of interest is PCR amplified to increase the amount of DNA to be detected. The most common detection technique with these systems is fluorescence, so the target DNA must be fluorescently tagged before it is flowed over the surface of the gene chip for detection. By viewing concentrated fluorescence signal caused by the hybridization of the target DNA to particular probes on the gene chip, and by knowing the nucleotide sequence of the probe molecules at those points of attachment, the presence of particular target molecules and their composition may be determined. These types of gene chips, which rely on fluorescence for the detection of complementary DNA sequences, are commercially available from companies such as Affymetrix and are widely used in the field of biotechnology. Other tagging methods including the use of radioactive substances may also be used, but is done so less frequently.

A promising technique that eliminates the need to use fluorescent or enzymatic tags is surface plasmon resonance (SPR). In SPR, a thin metallic film is illuminated from the backside “reflecting side” of the film. At a certain angle known as the plasmon angle, the energy from the illumination is coupled into electromagnetic waves creating a resonant condition (surface plasmon resonance) that is highly sensitive to surface conditions on the “sensing side” of the film opposite to the reflecting side.

SPR can be used in many different modes, such as SPR imaging, which allows multiple probe spots to be analyzed in parallel, so that arrays of different DNA probes may be attached to the surface. In the technique of SPR imaging, p-polarized light impinges on a prism, gold thin film, and flow cell assembly at a fixed angle. The reflected light is passed through a narrow band pass filter and collected by a CCD camera. In this technique probe molecules are covalently linked to discrete positions on the sensing side of the film to selectively bind with target molecules in the solution to be analyzed. The binding of the targets to the surface bound probes causes localized changes in the index of refraction at those spots, which are detected by the CCD camera. This data can then be used to identify the presence and composition of the target molecules by the location of the detected binding event.

While SPR imaging may eliminate the need for tagging target molecules, current SPR imaging methods can only detect DNA target molecules down to concentrations of approximately 10 nM. Accordingly time consuming and cumbersome amplification techniques such as PCR must be employed when looking at very low DNA copy numbers.

### SUMMARY OF THE INVENTION

The present invention provides a way to boost the sensitivity of SPR or other DNA detection techniques by as much as 10,000 times. The invention raises the possibility of direct detection of genomic DNA without the need for target amplification using PCR or the like.

Generally, the invention employs a mechanism for selectively destroying RNA probe molecules on a test surface when the RNA molecules have hybridized with target DNA in the sample being tested. When the RNA is destroyed, the previously hybridized DNA molecules may hybridize with new complementary RNA probe molecules initiating further cycles of destruction of complementary RNA probes. The ability of a single target DNA molecule to initiate the destruction of many complementary RNA probe molecules allows the detection of very low concentrations of DNA by observing RNA loss. This “intensification” effect, which does not require tagging or increasing the number of DNA target molecules when used with SPR imaging, can greatly simplify the detection of small concentrations of DNA.

Specifically then, the present invention provides a method of detecting target DNA molecules comprising the steps of introducing target DNA molecules into a test element holding RNA molecules complementary to the target DNA molecules and selectively destroying the RNA molecules that have hybridized with the target DNA molecules by an enzymatic process. The loss of RNA molecules caused by target DNA molecules successively hybridizing with different RNA molecules and their subsequent destruction by an enzymatic process is then detected.

It is one object of the invention to provide direct detection of extremely low concentrations of DNA.

The destruction of the RNA may be performed by the enzyme RNase H.

It is another object of the invention to provide a simple method for selectively destroying hybridized RNA suitable for use with the present invention.

The enzyme may be introduced into the test cell at the same time as the target DNA in single a solution.

Thus it is another object of the invention to provide for a convenient method of manipulating the necessary test sample and enzyme.

The test element may be a surface to which RNA molecules are attached.

Thus it is another object of the invention to provide a structure that may spatially support and segregate multiple different RNA molecules to provide for parallel analyses of DNA molecules comparable to that provided by conventional gene chips.

The surface may be selected from the group consisting of: diamond, glass, silicon, and gold.

Thus it is another object of the invention to provide a technique adaptable to a wide variety of detection technologies including SPR, fluorescent microscopy, and direct electrical detection.

The test element may be a surface plasmon resonance test cell, which consists of a microarray fabricated on a chemically modified gold surface and a flow cell, and the detection may be performed by an SPR imaging instrument for detecting the loss of RNA molecules from the test cell.

Thus it is one object of the invention to provide a system suitable for use with SPR instruments. It is another object of the invention to provide a detection system that avoids the need to tag the sample DNA.

Alternatively, the RNA molecules may be tagged with a fluorescent material and the detection step may be performed with a florescence detection apparatus, such as a fluorescence microscope or a fluoroimager.

It is thus another object of the invention to provide an intensification system applicable to a wide range of detection techniques.

These particular objects and advantages may apply to only some embodiments falling within the claims and thus do not define the scope of the invention.

### BRIEF DESCRIPTION OF THE FIGURES

FIG. 1 is a block diagram of a surface plasmon resonance imaging device suitable for use with the present invention, showing a test element allowing for the flow of target DNA and enzymes over a surface having attached RNA probes;

FIG. 2 is a plot showing a relationship between reflectance and incident angle in the SPR instrument for two concentrations of RNA probes at a particular site;

FIG. 3 is an elevational diagram showing the RNA probes attached to the substrate as may hybridize with a single stranded DNA target in solution with the enzyme RNase H;

FIG. 4 is a figure similar to that of FIG. 3 showing the destruction of a first hybridized RNA probe by RNase H, freeing the DNA for subsequent hybridization;

FIG. 5 is a figure similar to that of FIGS. 3 and 4 showing the destruction of a second hybridized RNA probe by RNase H, again freeing the DNA for subsequent hybridization;

FIG. 6 is a figure similar to that of FIGS. 3-5 showing completion of the destruction of the RNA probes on the test surface;

FIG. 7 is an elevational diagram of an alternative test surface having two different RNA probes, one complementary and one not complementary to a target DNA, and a DNA probe complementary to a target DNA;

FIGS. 8a-8c show plan images of the test surface of FIG. 7 at successive stages of hybridization and RNA hydrolysis;

FIG. 9 is a flow chart of the method of the present invention;

FIG. 10 is a planar view of a substrate providing probe molecules arranged in asymmetrical tile patterns for rotational identification;

FIG. 11 is a simplified representation of an alternative detection system using fluorescence sensitive scanning; and

FIG. 12 is a figure similar to FIG. 12 of an alternative detection system using radioactively tagged probes.

### DETAILED DESCRIPTION OF THE PREFERRED EMBODIMENT

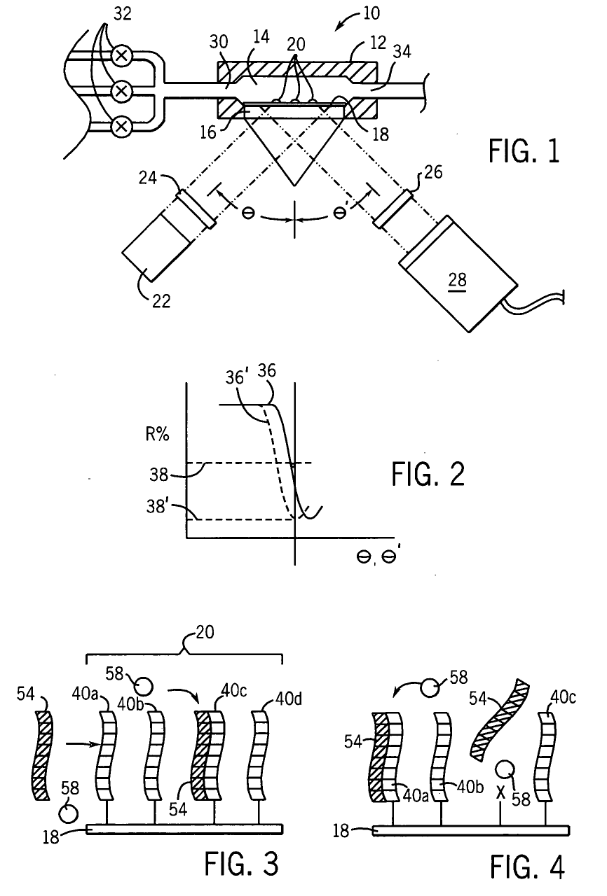

Referring now to FIG. 1, a surface plasmon resonance (SPR) device 10 may include a test element 12 providing a chamber 14. One wall of the chamber 14 provides a transparent test substrate 16 having on its inner surface, facing the chamber 14, a gold film 18. A front surface of the gold film 18 facing the chamber 14 may be spotted at probe locations 20 with molecules that may include RNA probe and control molecules and DNA control molecules in a regular pattern as will be described below.

In the SPR device, a collimated white light source 22, directs lights through a polarizer 24 at an angle q through a coupling prism to strike the rear surface of the gold film 18 after passing through the substrate 16. The light reflects off the gold film 18 at equal and opposite angle q′ to be received through a narrow band pass filter 26 by a CCD camera 28. The CCD camera 28 is focused on the rear surface of the gold film 18 to provide an image of the reflected light from that surface. As is understood in the art, the amount of material on the inner surface of the gold film 18 will affect the amount of reflected light received by the CCD camera 28 and thus, the image produced by the CCD camera 28, may be used to detect the amount of material on the front surface of the gold film 18. Systems suitable for this purpose are described in co-pending U.S. patent applications Ser. No. 10/411,583 filed Apr. 10, 2003, and Ser. No. 10/602,243 filed Jun. 24, 2003, assigned to the assignee of the present invention and hereby incorporated by reference.

The chamber 14 includes an inlet port 30 which may be connected by means of valves 32 to different fluid flow lines providing liquids for washing the surface of the front of gold film 18 and for introducing DNA samples and an enzyme into the chamber 14 as will be described. An exit port 34 allows liquids to be withdrawn from the chamber 14 and or recirculated.

Referring now to FIG. 2, reflectance 36 off the rear surface of the gold film 18 is a function of the angles q and q′ which may be adjusted to a fixed angle in order to produce a baseline reflectance 38 from the gold film 18 delineating the probe molecules at the probe locations 20 prior to reaction with sample DNA. A loss of material from the probe locations 20 will cause the reflectance curve to move to 36′ causing a significantly decreased reflectance at the fixed angle indicated by level 38′.

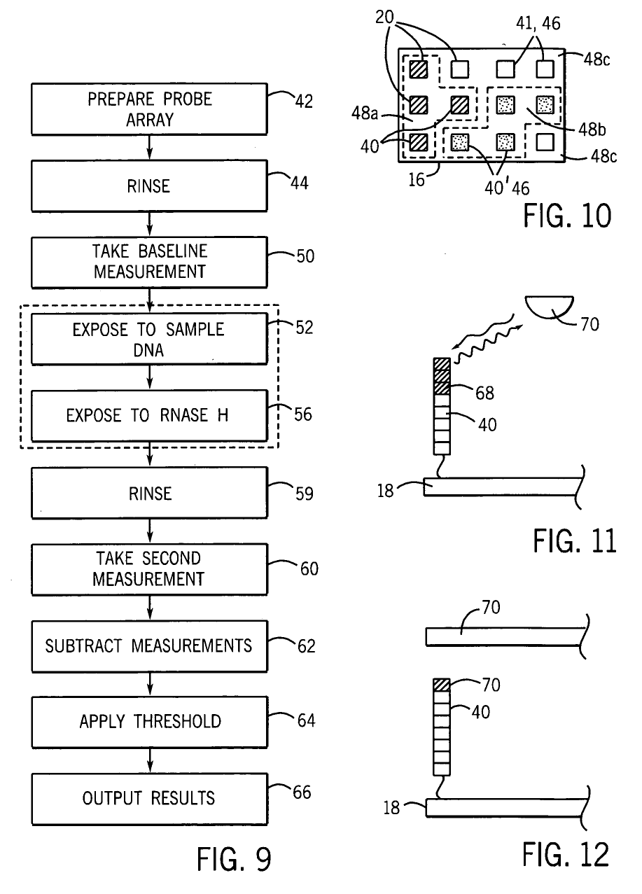

Referring now to FIGS. 3 and 9 per process block 42, RNA molecules 40 having a sequence of nucleotides complementary to an intended target DNA molecule may be covalently attached at predetermined probe locations 20 to the front surface of the gold film 18 to create an array. The RNA molecules 40 at each probe location 20 will have the same nucleotide sequence. In addition, and referring to FIG. 7, control molecules 46 being either RNA or DNA may be covalently attached to other predetermined probe locations 20 on the front surface of the gold film 18. The creation of the array on the gold film 18 containing RNA molecules 40 and the control molecules 46 and may use protection/de-protection surface chemistry and photo patterning adapted from Brockman, et al. and well known in the art. See generally, Brockman, J. M.; Frutos, A. G.; Corn, R. M.; J. Am. Chem. Soc. 1999, 121 8044-8051.

Referring momentarily to FIG. 10, the probe locations 20 may be square areas laid out in rectilinear columns and rows over the surface of the gold film 18. These probe locations 20 may be collected into zones 48a, 48b, and 48c, each having a common RNA molecule 40 or control molecule 46 so as to react similarly. The shapes of the zones 48a, 48b, and 48c are selected so as to be rotationally asymmetric so that the rotational orientation of the substrate 16 may be determined unambiguously by observation of the zones 48a-48c or any individual zone 48a-48c.

In an example embodiment, the probe locations 20 of zone 48a have RNA molecules 40 that will hybridize with an expected target DNA, while the probe locations 20 of zone 48b having RNA molecules 40′ that will not hybridize with the expected target DNA. The probe locations 20 of zone 48c, in contrast, may have DNA molecules that will hybridize with the target DNA. These latter two zones 48b and 48c provide control and calibration zones as will be described below. Alternatively, each of the zones 48a-48c may have RNA molecules 40 that will hybridize with different expected target DNA. The number of zones 48 and probe locations 20 in a zone may be freely varied.

Referring again to FIGS. 3 and 9, once the array is finished per process block 42, the slide is then mounted into the SPR imager and the front surface of the gold film 18 is rinsed per process block 44. All of the solutions used in the following steps may be autoclaved for sterilization. The rinsing process begins with water being washed over the gold film 18 to rinse away anything absorbed to the surface. Referring to FIG. 1, the water may be introduced through one of the valves 32. A buffer solution is then washed over the front surface of the gold film 18 made up of, for example, 50 mM Tris (pH 8.3), 50 mM KCl, and 10 mM MgCl2, 0.5 mM spermidine, and 10 mM DTT.

Referring still to FIG. 9, after the rinsing at process block 50, a first image of the gold film surface 18, using the SPR imaging instrument 10, may then be acquired. In this image, different probe locations 20 will have different reflectance and this reflectance may be measured and captured as a digital value related to the pixels associated with that portion of the image.

Next, at process block 52, a solution of 1 pM of the target DNA in 500 uL of the same buffer solution may rinsed over the gold film 18 introduced via one of the valves 32 shown in FIG. 1. This may be followed by the introduction of RNase H per process block 56, or per the preferred embodiment, 30 units (0.5 uL) of RNase H may be added to the 500 uL solution of DNA previously described so that the target DNA and enzyme can be present at the surface simultaneously. This process is not limited to only photopatterned arrays or large volume cells but may also make us of other techniques such as microfluidics.

Referring to FIG. 3 at this time, a target DNA molecule 54 may hybridize with one of the complementary RNA molecules 40c at a probe location 20.

Referring to FIG. 4, the RNase H 58 is an enzyme that has the property of hydrolyzing RNA molecules 40 only when an RNA molecule 40 is hybridized with a target DNA molecule 54. Thus as shown in FIG. 4, The RNase H 58 attacks the RNA 40c previously bonded to target DNA molecule 54, hydrolyzing the RNA molecules 40c and leaving the target DNA molecule 54 unharmed, free to react again with another RNA molecule 40. As also shown in FIG. 4, typically during the hydrolysis of one RNA molecule 40c, another DNA molecule 54 may be reacting with a different RNA molecule 40.

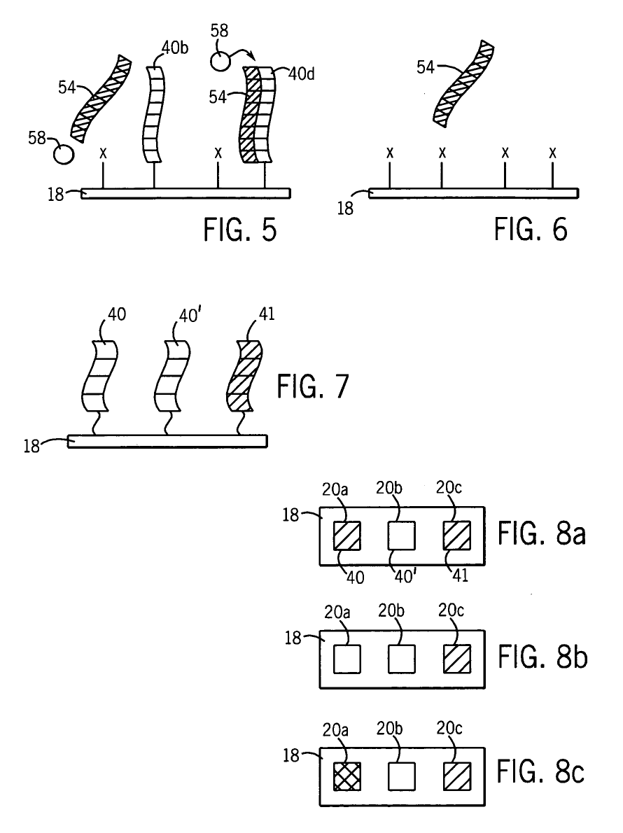

Referring now to FIG. 5, the DNA molecule 54 released in FIG. 4 after the hydrolysis of RNA molecule 40c, may hybridize with another RNA molecule 40d attached to the gold surface 18 while the RNA molecules 40a of FIG. 4, hybridized to target DNA molecule 54, is attacked by RNase H 58 to release its target DNA molecule 54 for subsequent hybridization.

As shown in FIG. 6, ultimately all of the RNA molecule 40a-40d complementary to the target DNA molecule 54 may be hydrolyzed and thus removed from the gold surface 18 in the given probe region 20. Because a single DNA molecule 54 may hydrolyze multiple RNA molecules 40, the effect of even a few DNA molecules is intensified. As an enzyme, the RNase H 58 is not consumed during the hydrolysis process. The present invention can provide a 10,000 times increase in sensitivity through this intensification process.

The solution of DNA molecules 54 and RNase H 58 is allowed to sit on the gold surface 18 for approximately thirty minutes to allow repeated hybridization and enzymatic hydrolysis of the RNA molecules 40 attached on the gold surface 18.

Referring again to FIG. 9, at the conclusion of the hydrolyzation process of process blocks 52 and 56 described above, the gold surface 18 is washed again with buffer, and as previously described, the washing as indicated by process block 59.

At succeeding process block 60, a second image of the gold film 18 is taken using the SPR instrument 10. In this image, those probe locations 20 where hydrolyses of RNA molecules will have different reflectance from the same probe locations in the image captured at previous process block 50. The reflectance of these probe locations are also captured digital values.

The arithmetic difference between the reflectance of given probe locations 20 may be determined by a computer receiving the two images according to techniques well known in the art as indicated by process block 62. Per process block 64, a threshold may be applied to the difference signal to determine which probe locations 20 have had reactions that have destroyed their RNA molecules 40 and thus which have encountered specific target DNA molecules 54. Experiments have determined that a perceptible difference in reflectance will occur for DNA target molecules in solution at concentrations down to 1 pM.

The output of this result may be indicated to the user through a subtraction image or an automated image processing system of types well known in the art as indicated by process block 66.

Referring now to FIG. 7, the gold surface 18 may include not only RNA molecules 40 intended to hybridize with target DNA molecules 54, but also RNA molecules 40′ intentionally of a sequence not binding with expected target DNA molecules 54 and DNA molecules 41 expected to hybridize with expected target DNA 54 which also hybridize with at least one of the RNA molecules 40. These RNA molecules 40′ and DNA molecules 41 serve as controls for monitoring the process, for example, to make sure the substrate is viable, and to provide a reference against which automatic measurement thresholds may be established.

For a gold surface 18 with control materials, the SPR instrument 10 is initially adjusted so that the reflectance of the gold surface 18 at the time of image of process block 50 of FIG. 9 is somewhere just off the SPR angle of curve 36 of FIG. 2 to provide a baseline reflectance 38.

Referring now to FIG. 8a, at the time of introduction of sample DNA molecules 54 of process block 52 of FIG. 9, the probe locations 20a and 20c will show increased reflectance caused by a binding of target DNA molecules 54 to the RNA molecules 40 and DNA molecules 41.

Referring to FIG. 8b, at the time of introduction of the RNase H 58 of process block 56 of FIG. 9, the probe location 20a will show decreased reflectance caused by a hydrolysis of the RNA molecules 40 while probe location 20c will not show this change.

At the time of the taking of a second image per process block 60 of FIG. 9, the reflectance of probe locations 20a will have dropped considerably below the reflectance of probe locations 20b and probe locations 20c caused by the loss of RNA molecules 40 in that probe location 20a. The reflectance of probe locations 20b or 20c may be used to establish a threshold used in process block 64 of FIG. 9 as described above.

While the present invention works well with SPR devices 10, which eliminates the need to tag the DNA molecules 54, the invention also may find application in a wide variety of other detection systems including, for example, those which detect the presence of the RNA probes 40 directly through electrical interaction with a treated silicon substrate.

The present invention also allows conventional fluorescent gene chip techniques to be used while still avoiding the need to tag the target DNA molecules 54. Referring to FIG. 11, in this case, the RNA molecules 40 may be tagged with a fluorescent dye 68. A conventional fluorescent scanner 70 of a type known in the art, may then stimulate the fluorescent dye 68 and record an image structurally similar to the SPR image at step 50 of FIG. 9. Upon completion of the hydrolysis, a second image may be obtained per block 60 of FIG. 9 and a subtraction image based on fluorescence rather than reflectance may be made to determine the loss of RNA molecules 40 caused by the hydrolysis process.

Referring to FIG. 12, alternatively, the RNA molecules 40 may be tagged with a radioactive material 70 and an image obtained by photographic film 72 or other techniques well known in the art.

Other techniques for detection of the loss of RNA molecules may also be used, and therefore, it will be understood that a wide variety of substrate materials may be used including glass and diamond.

It is specifically intended that the present invention not be limited to the embodiments and illustrations contained herein, but include modified forms of those embodiments including portions of the embodiments and combinations of elements of different embodiments as come within the scope of the following claims.

1. A method of detecting target DNA molecules comprising the steps of:
(a) introducing target DNA molecules into a test element holding RNA molecules complementary to the target DNA molecules;
(b) selectively destroying the RNA molecules that have hybridized with target DNA molecules; and
(c) detecting a loss of RNA molecules caused by the target DNA molecules successively hybridizing with different RNA molecules.

2. The method of claim 1 wherein step (b) introduces an enzyme into the test cell to destroy the hybridized RNA.

3. The method of claim 2 wherein the enzyme is RNase H.

4. The method of claim 1 wherein steps (a) and (b) are performed at the same time.

5. The method of claim 4 wherein step (b) introduces an enzyme into the test cell to destroy the hybridized RNA and wherein the target DNA and enzyme are mixed together in a solution introduced into the test cell.

6. The method of claim 1 wherein the test element is a surface to which the RNA molecules are attached.

7. The method of claim 6 wherein the surface is selected from the group consisting of: gold, diamond, glass, and silicon.

8. The method of claim 6 wherein the test element is a surface plasmon resonance (SPR) test cell to which the RNA molecules are attached, and step (c) is performed with an SPR imaging instrument detecting the loss of RNA molecules from the test cell.

9. The method of claim 8 wherein step (c) includes the steps of obtaining a baseline refraction measurement of the surface of the SPR test cell before step (b) and comparing the baseline refraction measurement to a refraction measurement of the surface of the SPR test cell after step (b).

10. The method of claim 8 wherein the SPR instrument produces an image of the SPR test cell and including the step before step (a) of attaching different RNA molecules to the surface in a predetermined pattern discernable in the image.

11. The method of claim 10 wherein the predetermined pattern is rotationally asymmetric.

12. The method of claim 1 wherein the SPR surface further includes control RNA that is not complementary to the target DNA and including the step of detecting the presence of the control RNA.

13. The method of claim 1 wherein the concentration of target DNA is less than 10 nM.

14. The method of claim 1 wherein the RNA molecules are tagged with a fluorescent material and wherein step (c) is performed with a fluorescence detection apparatus.

15. The method of claim 14 wherein step (c) includes the steps of obtaining a baseline fluorescence measurement of the surface of the RNA molecules of the test cell before step (b) and comparing the baseline fluorescence measurement to a fluorescence measurement of the RNA molecules of the test cell after step (b).

16. The method of claim 14 wherein the fluorescence detection apparatus produces an image of the test cell and including the step before step (a) of attaching the RNA molecules to a surface of the test cell in a predetermined pattern discernable in the image.

17. The method of claim 1 wherein the RNA molecules are tagged with a radioactive material and wherein step (c) is performed with a radiation detector.

18. The method of claim 14 wherein the radiation detector produces an image of the test cell and including the step before step (a) of attaching the RNA molecules to a surface of the test cell in a predetermined pattern discernable in the image.

19. The method of claim 16 wherein the predetermined pattern is rotationally asymmetric.

20. An apparatus for detecting target DNA molecules comprising:
a test element holding RNA molecules complementary to the target DNA molecules;
a port for introducing target DNA molecules into a test element;
a means for destroying the RNA molecules in the test cell selective to RNA molecules that have hybridized with target DNA molecules; and
a detector sensitive to a loss of RNA molecules caused the target DNA molecules successively hybridizing with different RNA molecules.

21. The apparatus of claim 18 wherein the means for destroying RNA molecules is an enzyme that selectively hydrolyses hybridized RNA.

22. The apparatus of claim 21 wherein the enzyme is RNase H.

23. The apparatus of claim 22 wherein the enzyme is mixed in solution with the target DNA for introduction through the port.

24. The apparatus of claim 20 wherein the test element is a surface to which the RNA molecules are attached.

25. The apparatus of claim 24 wherein the surface is selected from the group consisting of: gold, diamond, and silicon.

26. The apparatus of claim 24 wherein the test element is a surface plasmon resonance (SPR) test cell to which the RNA molecules are attached and the detector is an SPR imaging instrument detecting the loss of RNA molecules from the test cell.

27. The apparatus of claim 26 wherein the SPR instrument produces an image of the SPR test cell and wherein the RNA molecules include different RNA molecules attached to the surface in a predetermined pattern discernable in the SPR image.

28. The apparatus of claim 27 wherein the predetermined pattern is rotationally asymmetric.

29. The apparatus of claim 20 wherein the SPR surface further includes control RNA that is not complementary to the target DNA.

30. The apparatus of claim 20 wherein the RNA molecules are tagged with a fluorescent material and the detector is a fluorescence detection apparatus.

31. The apparatus of claim 30 wherein the fluorescence detection apparatus produces an image of the test cell and wherein the RNA molecules are attached to a surface of the test cell in a predetermined pattern discernable in the image.

32. The apparatus of claim 31 wherein the predetermined pattern is rotationally asymmetric.
